(19) United States
(12) Patent Application Publication
(10) Pub. No.: US 2008/0161782 A1
(43) Pub. Date: Jul. 3, 2008

(54) MICROPORE DELIVERY OF ACTIVE SUBSTANCES

(75) Inventors: Kin F. Chan, San Jose, CA (US); Basil M. Hantash, East Palo Alto, CA (US); Vikramaditya P. Bedi, Redwood City, CA (US); George Frangineas, Fremont, CA (US)

Correspondence Address: RELIANT / FENWICK;c/o FENWICK & WEST, LLP, 801 CALIFORNIA STREET, MOUNTAIN VIEW, CA, 94041, US

(73) Assignee: Reliant Technologies, Inc., Mountain View, CA (US)

(21) Appl. No.: 11/925,697

(22) Filed: Oct. 26, 2007

Related U.S. Application Data
(60) Provisional application No. 60/863,115, filed on Oct. 26, 2006.

(51) Int. Cl. A61B 18/20; A61K 31/341; A61P 17/00

(52) U.S. Cl. 606/9; 514/474

(57) ABSTRACT

A method and device for delivering active substances into and through the skin for treatment of the skin during and/or following fractional laser radiation treatment of the skin are described.

### CROSS-REFERENCE TO RELATED APPLICATION(S)

This application claims priority under 35 U.S.C. 119(e) to U.S. Provisional Patent Application Ser. No. 60/863,115, “Micropore Delivery of Active Substances”, filed Oct. 26, 2006. The subject matter of the foregoing is incorporated herein by reference in its entirety.

### TECHNICAL FIELD OF THE INVENTION

The present invention relates in general to methods and devices for delivering active substances into and through the skin following irradiation of the skin using ablative fractional laser radiation. More particularly, it relates to methods and devices for delivering compositions comprising an effective amount of an active substance in a carrier into the skin for local treatment of the skin during or following fractional ablative laser radiation treatment, where the fractional ablative laser treatment method provides a therapeutic and/or cosmetic treatment to the skin.

### INTRODUCTION

Various methods exist in the art for increasing the permeability of the skin so as to allow delivery of active substances into or through the skin. Chemical enhancers can be used to reduce the barrier function of the skin or to alter the properties of the active substance so as to allow the active substance to better partition into the skin. These chemical modifiers can be quite irritating to the skin, and may not increase permeability adequately to allow therapeutic levels of many active substances to permeate the skin.

Energy-driven methods of increasing skin permeability have been developed primarily for purposes of transdermal drug delivery, including electroporation and iontophoresis. Electroporation involves the use of relatively high electrical voltages over short periods of time to decrease the barrier function of the skin. Iontophoresis involves the use of relatively low electrical currents over a longer period of time to drive charged particles across the skin. Sonophoresis involves the use of ultrasound to drive active substances across the skin. The utility of these techniques is limited, as iontophoresis and electroporation are effective only with active substances that are stable in the presence of electrical currents, and all three methods increase skin permeability only during the period of time the treatment is applied.

Various methods of physically avoiding or removing the barrier function of the skin have also been used to increase permeability primarily for purposes of transdermal drug delivery. Microneedles, composed of arrays of very fine needles which pierce the upper layers of the skin to create holes through which active substances can penetrate, are considered minimally invasive. However, microneedles can be difficult to manufacture, and it can be difficult to position them within the skin so as to allow adequate permeation of active substances. Additionally, using microneedles can produce contaminated sharps, which pose a contamination threat and a medical waste disposal problem.

Various methods have been used to ablate the stratum corneum, the outermost or uppermost layer of the skin, which poses the greatest barrier to permeation for many active substances. Stratum corneum ablation techniques include suctioning, dermabrasion, and radiofrequency thermal ablation. Suctioning involves forming a small blister on the skin (usually with a vacuum), and removing the upper surface of the skin, thereby forming an area of skin without stratum corneum and allowing an active substance to readily permeate into and through the remaining skin layers. With suctioning, it is difficult to control the thickness of the blister created. Also, this technique produces relatively large areas of ablation that can take a long time to heal, resulting in an open portal for infection as well as active substances. As traditionally practiced, radiofrequency thermal ablation requires that an array of tiny, closely spaced electrodes be placed against the skin while an alternating current at radio frequency is applied to each microelectrode, thereby ablating the outermost layer of the skin. Control of the depth of ablation is difficult with this technique, and the need to place the microelectrodes directly in contact with the skin limits its utility.

Electromagnetic radiation, particularly as produced by lasers, has been applied directly to the skin for treatment of dermatological conditions, for skin resurfacing, to reduce or eliminate wrinkles, and to combat the effects of aging in the skin. Beyond treatment of the skin, electromagnetic radiation therapy has been used to increase the rate of wound healing, to reduce pain, to treat inflammatory conditions, as well as to reduce residual neurological deficits following stroke. When used for skin resurfacing, the effect of electromagnetic radiation on skin is primarily to heat the skin, producing coagulation, cell necrosis, melting, welding and ablation, among other effects. Treatment with electromagnetic radiation can generally be divided into ablative and nonablative treatments.

The use of nonablative electromagnetic irradiation of the skin has been suggested to increase skin permeability by altering the lipid and protein molecules present in the stratum corneum, by producing heat, and by producing pressure waves.

Ablation of the stratum corneum with electromagnetic radiation has been used for skin resurfacing and to perforate the skin to allow delivery of active substances and the removal or monitoring of biological fluids or gasses. U.S. Pat. No. 4,775,361 claims to describe a method of facilitating percutaneous transport by ablating the stratum corneum with pulsed laser radiation. The premise behind this invention is that the stratum corneum is the main barrier to permeation of active compounds, and the invention uses pulsed laser radiation to completely remove the barrier of the stratum corneum while avoiding penetration of the laser radiation into the viable epidermis.

United States Patent Application Publication Number US 2006/0004347 A1 discloses discusses methods of creating and differentiating types of “islets” in the skin, namely optical islets, thermal islets, damage islets, and photochemical islets. It reports that the creation of damage islets can be used to produce an increase in the permeability of the stratum corneum by heating islets of tissue to temperatures higher than 100° C. to create small holes in the stratum corneum. This application claims transdermal delivery methods which involve delivering portions of a topical preparation across the skin during the step of applying optical energy.

The methods described above focus on methods for delivering active ingredients through the skin for purposes of transdermal delivery. However, there remains a need for methods which deliver active ingredients such as vitamins into the skin for local treatment of the skin, including the promotion of healing of the skin following ablative laser treatments, such as those used for skin resurfacing and treatment of vascular lesions.

### SUMMARY OF THE INVENTION

The present invention is directed to compositions and methods of delivering active substances into and through the skin during and following fractional ablative laser treatments. Active substances are placed on the skin during the formation of the voids and/or following the formation of the voids to provide a therapeutic or cosmetic effect on the skin.

### BRIEF DESCRIPTION OF THE DRAWINGS

The accompanying drawings, which are incorporated in and constitute a part of the specification, schematically illustrate a preferred embodiment of the present invention, and together with the general description given above and the detailed description of the preferred embodiment given below, serve to explain principles of the present invention.

FIG. 1 is a micrograph of a section of human skin immediately after irradiation with laser radiation having parameters in accordance with the method of the present invention, the irradiated skin including a plurality of voids extending through the stratum corneum and the epidermis into the dermis, the voids being surrounded by regions of coagulated dermal tissue with viable tissue between the regions of coagulated tissue surrounding the voids.

FIG. 2 is a micrograph similar to the micrograph of FIG. 1 but having a lower magnification and depicting detail of the voids extending through the stratum corneum.

FIG. 3 is a micrograph of a section of human skin 48 hours after irradiation with laser radiation in having the parameters in accordance with the method of FIG. 1.

FIG. 4 is a micrograph of a section of human skin one week after irradiation with laser radiation in having the parameters in accordance with the method of FIG. 1.

FIG. 5 is a micrograph of a section of human skin one month after irradiation with laser radiation in having the parameters in accordance with the method of FIG. 1.

FIG. 6 is a graph schematically illustrating trend curves for maximum lesion or treatment zone with (void width plus coagulated tissue width) as a function of lesion or zone depth in the method of the present invention, for 5 mJ, 10 mJ, and 20 mJ pulses.

FIG. 7 is a graph schematically illustrating trend curves for maximum void width as a function of lesion or zone depth in the method of the present invention, for 5 mJ, 10 mJ, and 20 mJ pulses.

FIGS. 8A, 8B, and 8C are graphs schematically illustrating estimated width as a function of lesion or zone depth for lesions and voids with dimensions derived from micrographs of treatment sites in accordance with the present invention, for respectively 5 mJ, 10 mJ, and 20 mJ pulses.

FIG. 9A is a front elevation view schematically illustrating one example of apparatus suitable for irradiating skin according to the method of the present invention, the apparatus including a multi-faceted scanning wheel for scanning a pulsed, collimated laser beam and a wide field lens for focusing the scanned laser beam onto skin to sequentially ablate tissue and create the cauterized voids of the inventive method.

FIG. 9B is a front elevation view schematically illustrating further detail of beam focusing in the apparatus of FIG. 9A.

FIG. 9C is a side elevation view schematically illustrating still further detail of beam focusing in the apparatus of 9A.

FIG. 10 schematically illustrates detail of the scanning wheel of FIGS. 9A-C.

FIG. 11 schematically illustrates one example of a handpiece including the apparatus of FIGS. 9A-C, the handpiece including a removable tip connectable to a vacuum pump for exhausting smoke and ablation debris from the path of the laser beam.

### DETAILED DESCRIPTION OF THE INVENTION

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. The practice of the present invention will employ, unless otherwise indicated, conventional methods of preparative and analytical methods of chromatography, protein chemistry, biochemistry, recombinant DNA techniques and pharmacology, within the skill of the art.

The present invention provides compositions and methods for the delivery of one or more than one active substance into and/or through the skin during and/or following fractional laser treatment of the skin.

In one example, the active substance is delivered into the skin for local treatment of the skin. In another example, the active substance is a substance that promotes a local effect within the skin of a patient. In another example, the active substance is an active substance that promotes healing of the skin following treatment with fractional laser radiation. In yet another example, the cosmetically effective active substance can be selected from the group consisting of a vitamin, a mineral, an anti-oxidant, an agent that promotes skin recovery, and combinations thereof.

The vitamin can be selected from the group consisting of a provitamin, a vitamin cofactor, a vitamin derivative, a form of vitamin A, a carotenoid, a retinoid, a form of B complex vitamin, thiamin, vitamin B₁, riboflavin, nicotinic acid, vitamin B₆, pyridoxine, pyridoxal, pyridoxamine, pantothenic acid, biotin, vitamin B₁₂, a form of vitamin C, ascorbic acid, a form of vitamin D, a form of vitamin E, a tocopherol, a form of vitamin K, phylloquinone, a menanquinones, a form of carnitine, choline, folic acid, inositol, and combinations thereof. The vitamin can be selected from the group consisting of a form of vitamin C, a form of vitamin A, a form of vitamin E, and combinations thereof. The vitamin can be a form of vitamin C.

The mineral can be selected from the group consisting of a trace mineral, calcium, copper, fluoride, iodine, iron, magnesium, phosphorus, selenium, zinc, and combinations thereof.

The anti-oxidant can be selected from the group consisting of a vitamin, a mineral, a hormone, a carotenoid terpenoid, a non-carotenoid terpinoid, a flavonic polyphenolic, a phenolic acid, an ester of a phenolic acid, a non-flavinoid phenolic, citric acid, a lignan, a phytoestrogen, oxalic acid, phytic acid, bilirubin, uric acid, a form of lipoic acid, silymarin, a form of acetylcystine, an emblicanin antioxidant, a free-radical scavenger, a peroxiredoxin, a form of catalase, a form of superoxide dismutase (SOD), a form of glutathione, a form of thioredoxin, a form of coenzyme Q-10, a bioflavinoid, a green tea extract, epigallo catechin gallate (EGCG), and combinations thereof.

The agent that promotes skin recovery can be selected from the group consisting of an interleukin, a chemokine, a leukotriene, a cytokine, myeloperoxidase, an antibiotic, a growth factor, a heat shock protein, a matrix metalloproteinase, a hormone, an estrogen, tea tree oil, and combinations thereof.

In one example, the active substance is delivered into the skin for local treatment of the skin to reduce the appearance of wrinkles in the skin and to reduce the effects of ageing of the skin. In another example, the active substance is selected from the group consisting of a vitamin, a mineral, an anti-oxidant, an agent to promote recovery, a growth factor, a cytokine, a heat shock protein, an agent to induce collagen remodeling, paeoniflorin, a form of alpha hydroxyl acid, a form of beta hydroxyl acid, a form of kinetin, a retinoid, a form of emu oil, a form of ubiquinone, a humectant, a neurotoxin, a muscle relaxant, and combinations thereof.

The active substance can be a retinoid. The retinoid can be selected from the group consisting of vitamin A, retinol, retinoic acid, tretinoin, isotreninoin, alitretionoin, etreinate, acitretin, an arotinoid, tazarotene, bexarotene, adapalene, Ro 13-7410, Ro15-1570, and combinations thereof.

The active substance can be a neurotoxin. The neurotoxin can be selected from the group consisting of a neurotoxic compound produced by a form of Clostridia, a neurotoxic compound produced by Clostridium botulinum, a form of botulinum toxin, botulinum toxin type A, botulinum toxin type B, botulinum toxin type C, botulinum toxin type D, botulinum toxin type E, botulinum toxin type F, botulinum toxin type G, a botulinum neurotoxin peptide, a botulinum neurotoxin A (BoNT/A) peptide, a botulinum toxin in combination with a polysaccharide, a botulinum toxin in combination with a carrier comprising a polymeric backbone having attached positively charged branching groups, a botulinum toxin in combination with human serum albumin, a botulinum toxin in combination with a neuron growth inhibitor, a botulinum toxin in combination with a non-oxidizing amino acid derivative and zinc, a botulinum toxin in combination with a recombinant gelatin fragment, a stabilized botulinum toxin composition, and combinations thereof.

In one example, the active substance is delivered into the skin for local treatment of the skin to reduce inflammation and the discharge of fluid from the skin during and following the laser treatment. In another example, the active substance is selected from the group consisting of a glucocorticoid, an antihistamine, an anti-inflammatory, a vasoconstrictor, and combinations thereof. In another example, the active substance is a substance that promotes local vasoconstriction within the skin of a patient.

The active substance can be a glucocorticoid. The glucocorticoid can be selected from the group consisting of betamethasone, betamethasone diproprionate, betamethasone valerate, clobetasol propionate, difluorasone diacetate, halobetasol propionate, actinomine, desoximetasone, fluocinonide, fluocinolone acetonide, flurandrenolide, hydrocortisone, hydrocortisone butyrate, hydrocortisone valerate, halcinonide, triamcinolone acetonide, amcinonide, mometasone furoate, aclometasone dipropionate, desonide, dexamethasone, dexamethasone sodium phosphate, and combinations thereof.

The active substance can be an antihistamine. The antihistamine can be selected from the group consisting of doxepin hydrochloride, caribinoxamine maleate, clemastine fumarate, diphenhydramine hydrochloride, dimenhydrinate, pyrilaimine maleate, tripelennamine hydrochloride, tripelennamine citrate, chlorpheniramine maleate, brompheniramine maleate, hydroxyzine hydrochloride, hydroxyzine pamoate, cyclizine hydrochloride, cyclizine lactate, meclizine hydrochloride, promethazine hydrochloride, cyproheptadine hydrochloride, phenindamine tartrate, acrivastine, cetirizine hycrochloride, azelastine hydrochloride, lovocasastine hydrochloride, loratidine, fexofenadine, and combinations thereof.

The active substance can be an anti-inflammatory drug. The anti-inflammatory drug can be selected from the group consisting of histamine, a histamine antagonist, bradykinin, a bradykinin antagonist, a lipid-derived autacoid, an eicosanoid, a platelet-activating factor, an analgesic-antipyretic agent, a cyclooxygenase-2 (COX-2) inhibitor, a drug for treatment of gout, a drugs for treatment of asthma, and combinations thereof. The anti-inflammatory agent can be a non-specific COX-2 inhibitor. The non-specific COX-inhibitor can be a salicylic acid derivative, aspirin, sodium salyclate, choline magnesium trisalicylate, salsalate, diflunisal, sulfasalazine, olsalazine, a para-aminophenol derivative, acetaminophen, an indole, an indene acetic acid, indomethacin, sulindac, a heteroaryl acetic acid, tolmetrin, diclofenac, ketorolac, a arylpropionic acid, ibuprofen, naproxen, flurbiprofen, ketoprofen, fenoprofen, oxaproxin, an anthranilic acid, mefenamic acid, meclofenamic acid, an enolic acid, an oxicam, proxicam, meloxicam, an alkonone, nabumetone, and combinations thereof. The anti-inflammatory agent can be a selective COX-2 inhibitor selected from the group consisting of a diaryl-substituted furanone, a diaryl-substituted pyrazole, an indole acetic acid, a sulfonanilide, and combinations thereof. The COX-2 inhibitor can be selected from the group consisting of celecoxib; rofecoxib; meloxicam; piroxicam; valdecoxib, parecoxib, etoricoxib, CS-502, JTE-522; L-745,337; FR122047; NS398; from non-selective non-steroidal anti-inflammatory agents that would include aspirin, ibuprofen, indomethacin CAY10404, diclofenac, ketoprofen, naproxen, ketorolac, phenylbutazone, tolfenamic acid, sulindac, and others, or from steroids or corticosteroids. Compounds which selectively inhibit cyclooxygenase-2 have been described in U.S. Pat. Nos. 5,380,738, 5,344,991, 5,393,790, 5,466,823, 5,434,178, 5,474,995, 5,510,368 and WO documents WO96/06840, WO96/03388, WO96/03387, WO95/15316, WO94/15932, WO94/27980, WO95/00501, WO94/13635, WO94/20480, and WO94/26731, and are otherwise known to those of skill in the art.

The active substance that can be delivered by the fractional laser treatments described herein can be a vasoconstrictor. The vasoconstrictor can be selected from the group consisting of an antihistamine, a form of adrenaline, a form of asymmetric dimethylarinine, a form of adenosine triphosphate (ATP), a catecholamine, cocaine, a decongestant, a form of diphenhydramine, a form of endothelin, a form of phenylephrine, a form of epinephrine, a form of pseudoephedrine, a form of neuropeptide Y, a form of norepinephrine, a form of tetrahydrozoline, a form of thromboxane, and combinations thereof.

In one example, the active substance is delivered into the skin for local treatment of the skin to reduce the likelihood of infection following the laser treatment. In another example, the active substance is selected from the group consisting of an antimicrobial compound, an antifungal compound, an antiviral compound, an antibiotic compound, and combinations thereof.

The antimicrobial compound can be selected from the group consisting of a sulfonamide, trimethoprim-sulfamethoxazole, a quinolone, a drug for treatment of urinary tract infections, a penicillin, a cephalosporin, a β-lactam antibiotic, an aminoglycoside, a protein synthesis inhibitor, and combinations thereof.

The antifungal compound can be selected from the group consisting of an azole, fluconazole, ketaconazole, micronazole, itraconazole, econazole, econazole nitrate, an allylamine, naftifine, terbinafine, griseofulvin, ciclopirox and combinations thereof.

The antiviral compound can be selected from the group consisting of acyclovir, famciclovir, valacyclovir, penciclovir, podophyllin, podofilox, imiquimod and combinations thereof.

The antibiotic compound can be selected from the group consisting of tetracycline, doxycycline, minocycline, erythromycin, trimethoprim, sulfamethoxazole, clindamycin, mupirocin, silver sulfadiazine, and combinations thereof.

In one example, the active substance is delivered into the skin to induce local anesthesia in the skin. In another example, the active substance is a local anesthetic. The local anesthetic can be selected from the group consisting of benzocaine, bupivicaine, chloroprocaine, cocaine, etidocaine, lidocaine, mepivacaine, pramoxine, prilocalne, procaine, proparacaine, ropivicaine, tetracaine, and combinations thereof.

In one example, the active substance is delivered into the skin for local treatment of acne. In another example, the active substance is a drug for treatment of acne. The drug for treatment of acne can be selected from the group consisting of azelaic acid, benzoyl peroxide, clindamycin, erythromycin, tetracycline, trimethoprim, minicycline, doxycycline, metronidazole, sulfacetamine, sulfur, salicylic acid, a retinoid, spironolactone, cyproterone acetate, a glucocorticoid, an estrogen, a progestin, prednisone, dexamethasone, and combinations thereof.

In one example, the active substance is delivered into the skin for treatment of alopecia. In another example, the active substance is a drug to treat alopecia. The drug to treat alopecia can be selected from the group consisting of a calcium channel blocker, minoxidil, a 5-alpha reductase inhibitor, finasteride, dutasteride, a retinoid, and combinations thereof.

In one example, the active substance of the invention is a composition comprised of an effective amount of an active substance in a carrier. In another example, the composition is comprised of a cosmetically effective amount of an active substance in a cosmetically acceptable carrier. In another example, the composition comprises a semi-solid. In another example, the composition comprises a lotion, cream, gel or ointment. In another example, the composition comprises a mask. In another example, the composition comprises a hydrogel mask. In yet another example, the composition comprises a urethane foam.

In one example, laser ablation forming the spaced-apart voids causes the voids to be surrounded with coagulated tissue immediately following the irradiation. There is viable tissue remaining between the voids. The coagulated tissue is under tension resulting from collagen shrinkage by heat generated during the abrasion process. The tension in the coagulated tissue shrinks the voids. The active substance is deposited into the voids. A healing process completely replaces the coagulated tissue with new tissue after a period of about one month.

In another example, the present invention provides an apparatus for delivering active substances into the skin of in a subject in need thereof, the apparatus comprises a handpiece movable over skin wherein the handpiece is arranged to receive an optical beam and focus the optical beam at a plurality of spaced-apart locations on the skin thereby creating a plurality of voids in the skin for the deposition of a composition. In yet another example, the composition deposited by the apparatus comprises a cosmetically effective amount of an active substance in a cosmetically acceptable carrier.

In one example, the compositions are applied to one or more micropore channel(s) or void(s) in the skin, wherein the micropore channel(s) or void(s) can be created using laser irradiation of the skin. The micropore channel or void preferably extends through the stratum corneum and the epidermis into the dermis and is surrounded by regions of coagulated dermal tissue. Preferably viable tissue is present between adjacent micropore channels or voids. The viable tissue promotes healing of the treatment zones.

The active substances described above can be delivered into the skin for local treatment of the skin and promoting recovery of the skin after the skin has been treated using fractional ablative laser therapy for a variety of purposes, including, but not limited, fractional ablative laser skin resurfacing treatments, treatment of wrinkles using fractional ablative laser techniques, treatment of photoaging of the skin using fractional ablative laser techniques, treatment of vascular lesions using fractional ablative laser techniques, and laser-assisted hair transplant therapy.

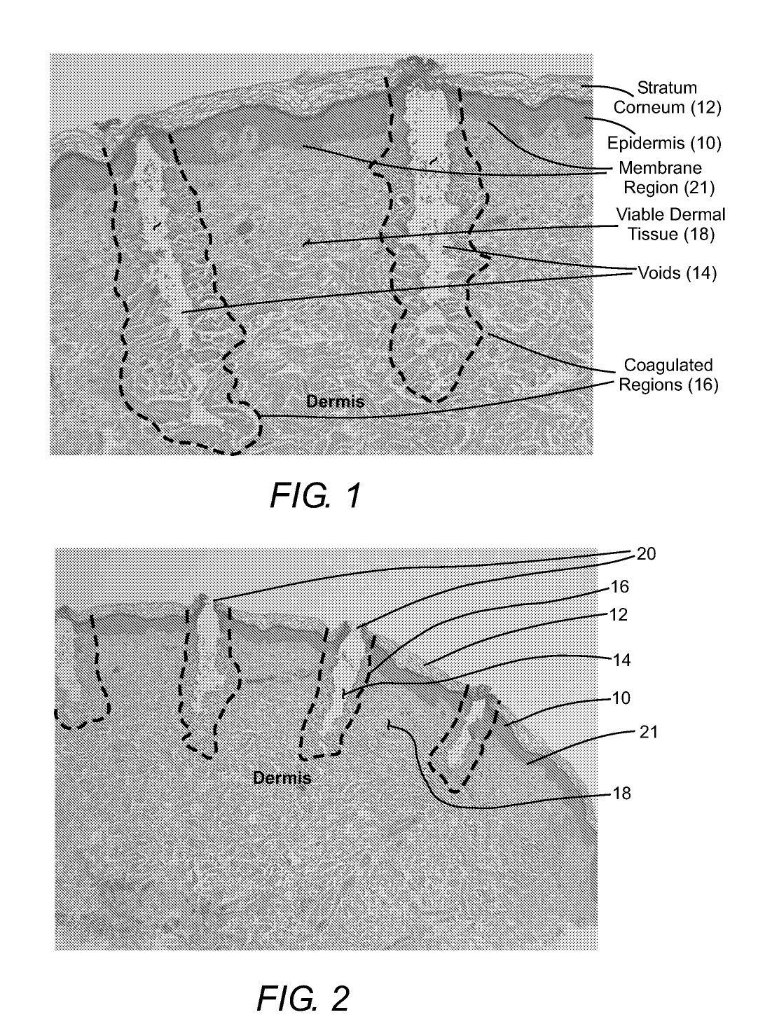

Referring now to the drawings, wherein like features are designated by like reference numerals, FIG. 1 and FIG. 2 are micrographs schematically illustrating a section of human skin immediately after immediately after irradiation with laser radiation to provide microchannels or voids capable of receiving a vitamin in accordance with the method of the present invention. FIG. 2 is at twice the magnification of FIG. 1. The skin was irradiated at spaced-apart locations with pulses of radiation having a wavelength of 10.6 micrometers (μm) from a CO₂ laser delivering a substantially TEM₀₀-quality beam. Each location was irradiated by one pulse. The radiation at the locations was focused to a spot having a diameter of about 120 μm at the surface of the skin, expanding slightly to between about 150 μm and 170 μm at a depth of about 1 mm in the skin. The laser output was repetitively pulsed at a pulse repetition frequency (PRF) of about 60-100 Hz. The pulses were nominally “square” laser pulses having a peak power of about 40 Watts (W) and a pulse duration of about 0.5 milliseconds (ms) to produce a pulse energy of 20 millijoules (mJ). The pulse duration could be varied to create different pulse energies for other experimental treatments. Experimental evaluations were performed with pulse energies in a range between about 5 mJ and 40 mJ. Laser pulses were scanned over the surface using a scanner wheel device to provide the spaced apart voids. The PRF of the laser was synchronized with the rotation of the scanner wheel. A detailed description of a preferred example of such a scanner wheel is presented further herein below.

The skin tissue includes a bulk dermal portion or dermis covered by an epidermal layer (epidermis) 12 typically having a thickness between about 30 μm and 150 μm. The top layer of the epidermis is covered, in turn, by a stratum corneum layer 10 typically having a thickness between about 5 μm and 15 μm. Tissue was ablated at each pulse location, producing a plurality of spaced-apart voids 14, elongated in the direction of incident radiation, and extending through the stratum corneum and the epidermis into the dermis.

In the example of FIGS. 1 and 2, the voids with the parameters mentioned above have an average diameter (width) of between about 180 μm and 240 μm. These dimensions are provided merely for guidance, as it will be evident from the micrographs that the diameter of any one void varies as the result of several factors including, for example, the inhomogeneous structure and absorption properties of the tissue. The voids have an average depth of between about 800 μm and 1000 μm, and are distributed with a density of approximately 400 voids per square centimeter (cm²). Walls of the voids are substantially cauterized by heat generated due to the ablation, thereby minimizing bleeding in and from the voids. This heat also produces a region 16 of coagulated tissue (coagulum) surrounding each void. Note that the term “surrounding” as used in this application does not imply that there is tissue remaining above the void. Here, the void is defined as being surrounded by coagulated tissue if dermal tissue around the walls of the void is coagulated. The void is defined as the region that is ablated. Immediately following ablation the voids are open. The appearance of closure of some voids in FIG. 2 is believed to be an artifact of the preparation of tissue samples for microscopic evaluation.

The coagulated regions have a thickness between about 20 μm and 80 μm immediately after ablation of the voids. Here again, however, thickness varies randomly with depth of the void because of above-mentioned factors affecting the diameter of the void. Between each void 14 and the surrounding coagulum 16 is a region of 18 of viable tissue. This includes a viable region of the stratum corneum, the epidermis, and the dermis. Preferably the region of viable tissue has a width, at a narrowest point thereof, at least about equal to the maximum thickness of the coagulated regions 16 to allow sufficient space for the passage of nutrients to cause rapid healing and to preserve an adequate supply of transit amplifying cells to perform the reepithelialization of the wounded area. More preferably, the viable tissue separating the coagulated tissue around the voids has a width, at a narrowest point thereof, between about 50 μm and 500 μm. A preferred density of treatment zones is between about 200 and 4000 treatment zones per cm². The density of treatment zones can be higher than the desired hair density because not every stem cell implantation sites will produce a viable hair follicle. This treatment-zone density can be achieved in a single pass or multiple passes of a treatment device of applicator, for example two to ten passes, in order to minimize gaps and patterning that may be present if treatment zones are created in a single pass of the applicator.

Heat from the ablation process that causes the coagulation in regions 16 effectively raises the temperature of the collagen in those coagulated regions sufficiently to create dramatic shrinkage or shortening of collagen in the coagulated tissue. This provides a hoop of contractile tissue around the void at each level of depth of the void. Upon collagen shrinkage, the dermal tissue is pulled inward, effectively tightening the dermal tissue. This tightening pulls taut any overlying laxity through a stretching of the epidermis and stratum corneum. This latter response is primarily due to the connection of a basement membrane region 21 of the epidermis to the collagen and elastin extra-cellular matrix. This connection provides a link between the epidermis and dermis. The contractile tissue very quickly shrinks the void, and creates an increase in skin tension resulting in a prompt significant reduction in overall skin laxity and the appearance of wrinkles. This shrinkage mechanism is supplemented by a wound-healing process healing described below.

Closure of the void occurs within a period of about 48 hours or less through a combination of the above-described prompt collagen shrinkage and the subsequent wound healing response. The wound healing process begins with re-epithelialization of the perimeter of the void, which typically takes less than 24 hours, formation of a fluid filled vacuole, followed by infiltration by macrophages and subsequent dermal remodeling by the collagen and elastin forming fibroblasts. The column of coagulated tissue has excellent mechanical integrity that supports a progressive remodeling process without significant loss of the original shrinkage. In addition, the coagulated tissue acts as a tightened tissue scaffold with increased resistance to stretching. This further facilitates wound healing and skin tightening. The tightened scaffold serves as the structure upon which new collagen is deposited during wound healing and helps to create a significantly tighter and longer lasting result than would be created without the removal of tissue and the shrinkage due to collagen coagulation.

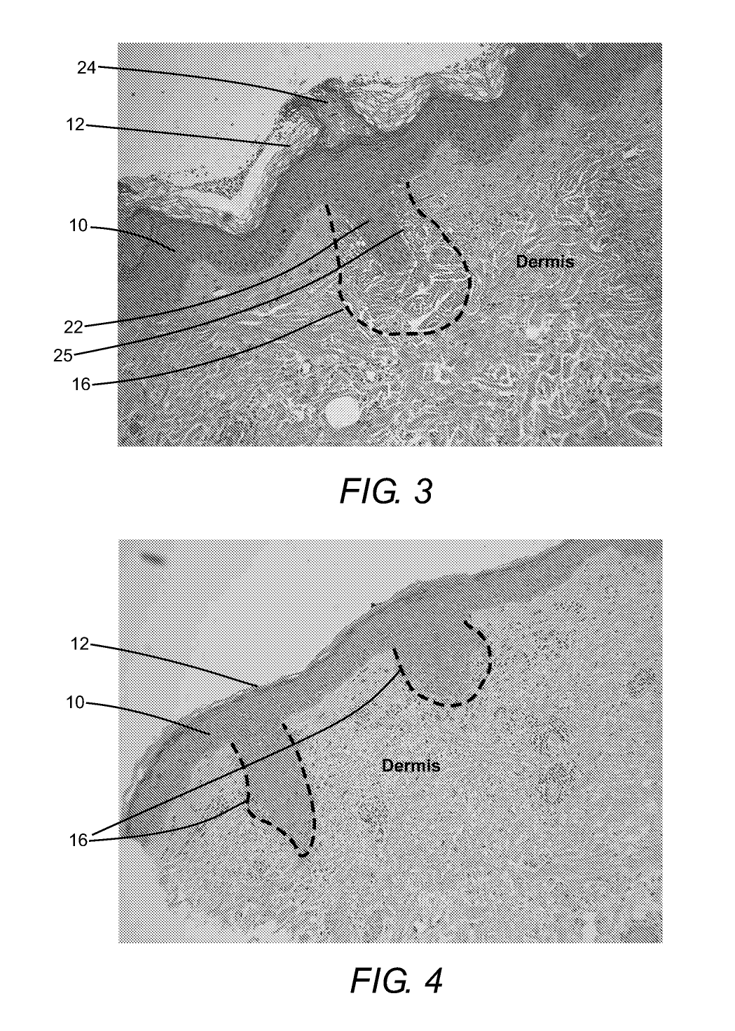

Progress of the healing after a period of about 48 hours from the irradiation conditions of FIG. 1 is illustrated by the micrograph of FIG. 3, which has the same magnification. Here, the coagulated region 16 is reduced both in diameter and depth compared with a comparable region of FIG. 1. In the micrograph of FIG. 3 epidermal stem cells have migrated into the void and facilitated healing of the void area. Epidermal stem cells proliferate and differentiate into epidermal keratinocytes filling the void in a centripetal fashion. As epidermal cells proliferate and fill the void, the coagulated material is pushed up the epidermis toward the stratum corneum. The voids contain microscopic-epidermal necrotic debris (MEND). The pushing of the coagulated material forces a plug 24 of the MEND to seal the stratum corneum during the healing response, thus preventing access of the outside environment to the inside of the skin.

At this time, the basement membrane is ill-defined and has yet to be completely repaired and restored. This is clearly depicted by the vacuolar space 25 separating the healed void and the dermis. In FIGS. 1 and 2, there is sparse cellularity evident in the dermis. However, in the micrograph of FIG. 3, the wound healing response at 48 hours has led to increased release of signaling molecules, such as chemokines, from the area of spared tissue, leading to recruitment of inflammatory cells aiding in the healing response.

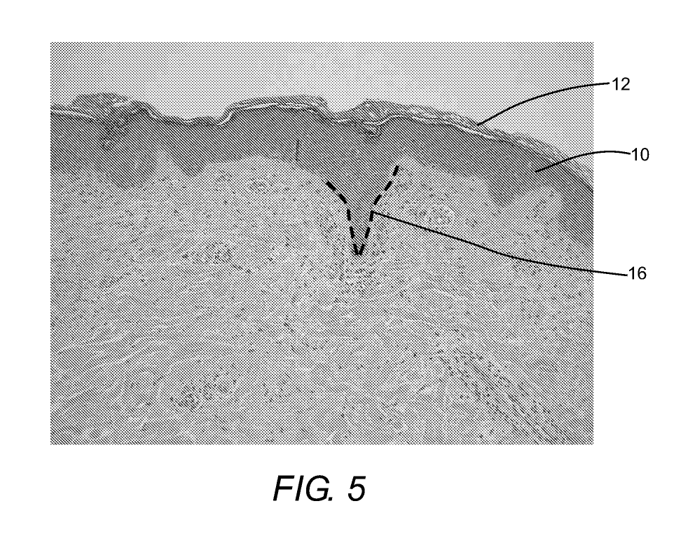

Progress of the healing after a period of about one week from the irradiation conditions of FIG. 1 is illustrated by the micrograph of FIG. 4. Here, the MEND has been exfoliated. The void has been replaced by epidermal cells which gradually remodel to create a normal rete ridge pattern, reducing in depth of invagination. The healing process has triggered that some of the deeper epidermal cells go through apoptosis, thereby disappearing from the replaced void tissue. The basement membrane of the epidermis has almost fully been restored as evidenced by the lack of vacuolization between the epidermis and dermis. During the wound healing response, cytokines such as TGF beta, amongst others, are released and allow fibroblasts to secrete collagen, elastin, and extracellular matrix. This secreted matrix replaces the apoptotic epidermal cells of the void. The coagulated dermal tissue has been replaced by a similar process sparked by the laser irradiation treatment induced release of pro-neocollagenesis cytokines. Inflammatory cells also help remove non-viable debris in the dermis, allowing the replacement of coagulated tissue with fresh viable tissue as outlined above.

FIG. 5 depicts progress of healing one month after initial treatment. Here remodeling of the void has continued by apoptosis of the deeper epidermal cells, leading to a more natural rete ridge like structure. The MEND is absent, and the basement membrane of the epidermis is completely healed. Inflammatory cells are still present in the dermis, and fibroblasts continue to lay down new matrix in the dermis. This provides that over the ensuing two to six months, new collagen synthesis continues to replace previously coagulated dermal tissue, providing for increased tensile strength in the dermis.

The complete replacement of the coagulated tissue providing the initial skin tightening with new collagen and elastin as described above provides for a long lasting improvement in the appearance of wrinkles in temporally or photo aged skin. As the inventive method results in a completely healthy treated area once the healing process is complete, an area of skin treated once can be treated again, for example, after a period of about two months to provide further improvement. Clearly, however, the progress of skin aging and loosening can not be arrested permanently, and the length of time that any improved appearance will be evident will depend on the age of the person receiving the treatment and the environment to which treated skin is exposed, among other factors.

In the example described above, skin irradiation for void formation is performed with laser radiation having a wavelength (10.6 μs) that is strongly absorbed by water. Preferably the radiation is delivered as a beam having TEM₀₀ quality, or near TEM₀₀ quality. The CO₂ laser used in the example of the present invention discussed above is a relatively simple and relatively inexpensive laser for providing such a beam. The 10.6 μm radiation of a CO₂ laser has an absorption coefficient in water of approximately 850 inverse centimeters (cm⁻¹). To efficiently ablate tissue, a high absorption coefficient in the water of the skin tissue is desired. However, in order to form a coagulation region surrounding the voids, to cause tissue shrinkage and to reduce bleeding at the treatment sites, the absorption coefficient should not be too high. Preferably, laser radiation used in the inventive method should have an absorption coefficient in water in the range between about 100 cm⁻¹ and 12,300 cm⁻¹. More preferably, the absorption coefficient should be between about 100 cm⁻¹ and 1000 cm⁻¹ and more preferably in the range between about 500 cm⁻¹ and 1000 cm⁻¹. In each of these absorption levels, laser pulses for forming the voids preferably have a duration of between about 100 microseconds (μs) and 5 milliseconds (ms). The actual treatment parameters can be chosen based on commercial tradeoffs of available laser powers and desired treatment-zone sizes. Lasers providing radiation having a wavelength that has an absorption coefficient in water in the preferred ranges include CO₂, CO, and free-electron lasers (500-1000 cm⁻¹), thulium-doped fiber lasers and free-electron lasers (100-1000 cm⁻¹), Er:YAG lasers, Raman-shifted erbium-doped fiber lasers, and free-electron lasers (between about 100 cm⁻¹ and 12,300 cm⁻¹). Other light sources, such as optical parametric oscillators (OPOs) and laser pumped optical parametric amplifiers (OPAs) can also be used.

Voids 14 preferably have a diameter between about 100 μm and 500 μm, and are preferably spaced apart with a center to center distance of between about 200 μm and 1500 μm depending on the size of the voids 14 and the coagulated regions 16. The center to center distance can be chosen based on the level of desired treatment. A coverage area for the coagulated regions and voids immediately following treatment is preferably between about 5% and 50% of the treated area. A higher level of coverage will be more likely to have a higher level of side effects for a similar treatment energy per treatment site. A preferred depth of the voids is between about 200 μm and 4.0 millimeters (mm). The voids are preferably randomly distributed over an area of skin being treated.

In relative and practical terms, the voids are preferably placed such that coagulated zones 16 surrounding the voids are separated by at least the average thickness of the coagulated zones. This can be determined by making micrographs of test irradiations, similar to the above-discussed micrographs of FIGS. 1 and 2. If voids are too closely spaced, the healing process may be protracted or incomplete. If voids are spaced too far apart, more than one treatment may be necessary to achieve an acceptable improvement. Regarding depth of the voids, the voids and surrounding coagulated zones must extend into the dermis in order to provide significant skin tightening. The voids should preferably not, however, completely penetrate the skin or extend into subcutaneous fatty tissue.

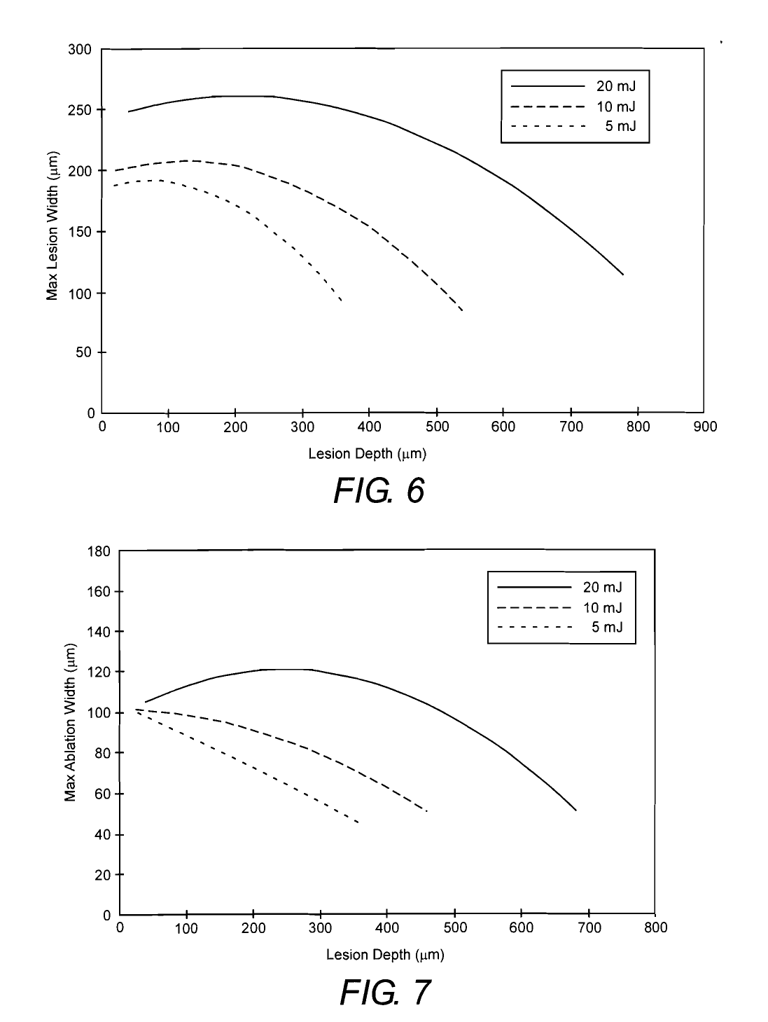

FIG. 6 and FIG. 7 are graphs schematically illustrating respectively trends for maximum width of the a treatment zone (lesion), i.e., maximum total width of a void 14 plus surrounding coagulated region 16, and maximum width of the void (ablated region), as a function of lesion depth, i.e., the depth to the base of the coagulated region. The trends in each graph are shown for pulse energies of 5 mJ, 10 mJ, and 20 mJ. It should be noted here that these trends fitted through a number of experimental measurements with relatively wide error bars, particularly at shallow lesion depth. Accordingly, it is recommended that these graphs be treated as guidelines only.

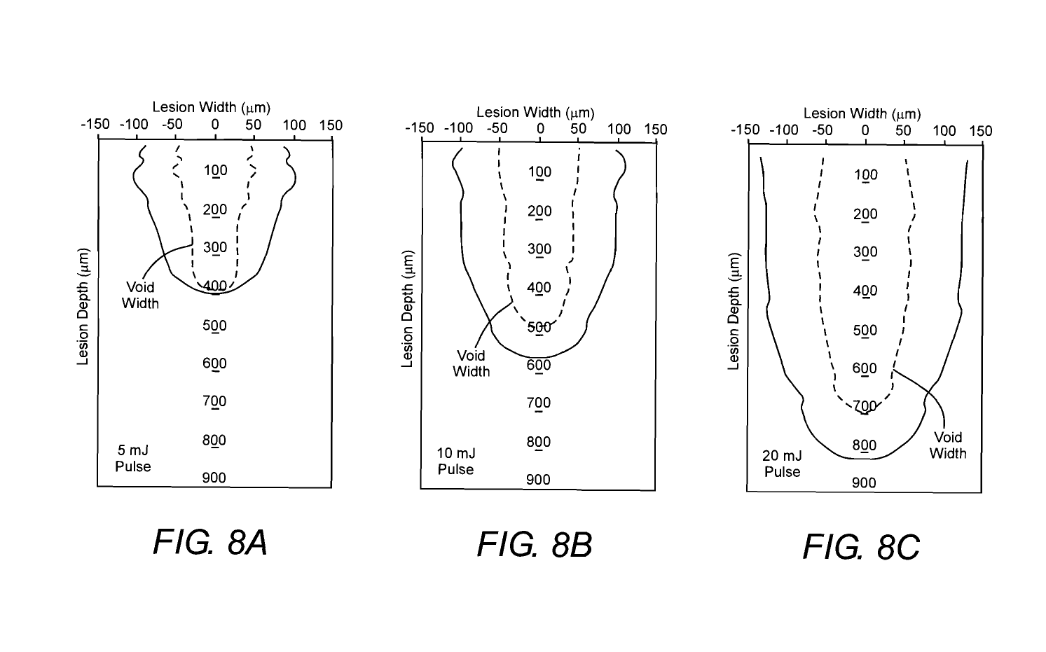

FIG. 8A, FIG. 8B, and FIG. 8C are graphs schematically illustrating graphical lesion width (solid curves) and void width (dashed curves) as a function of lesion depth for experimental irradiations at respectively 5 mJ, 10 mJ, and 20 mJ. These graphs are derived from measurements taken from micrographs of transverse sections through the experimental legions. The graphs of FIGS. 7 and 8A-C can be used as guidelines to select initial spacing of treatment zones in the inventive method. This spacing can then be optimized by experiment.

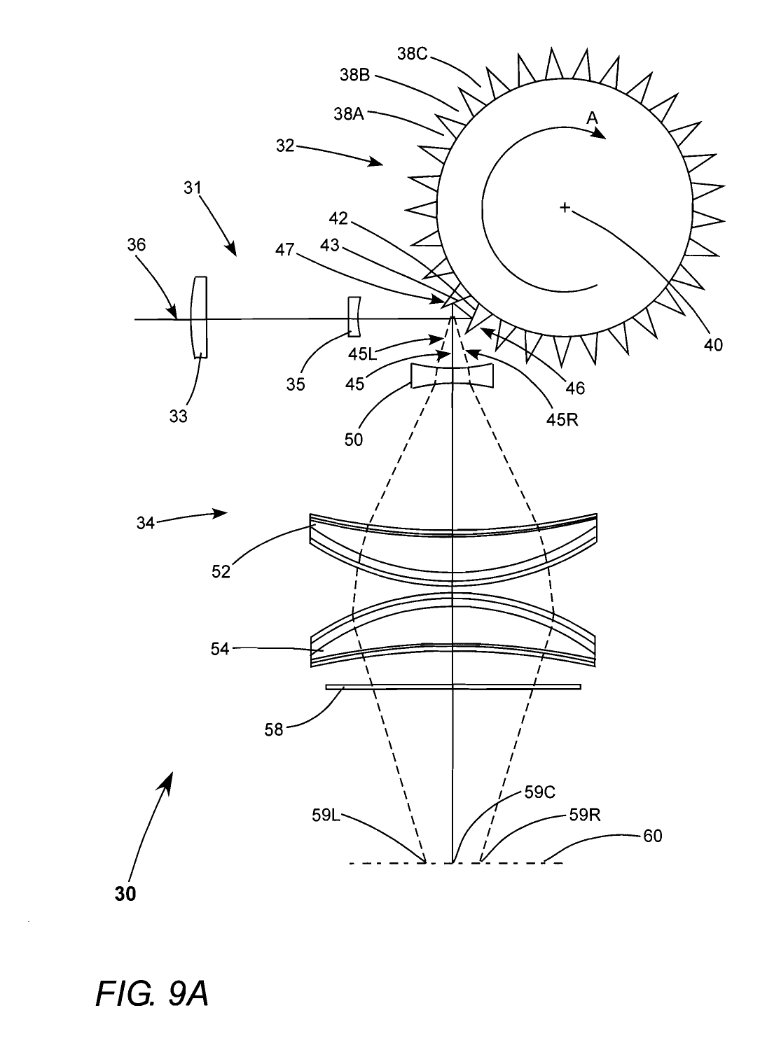

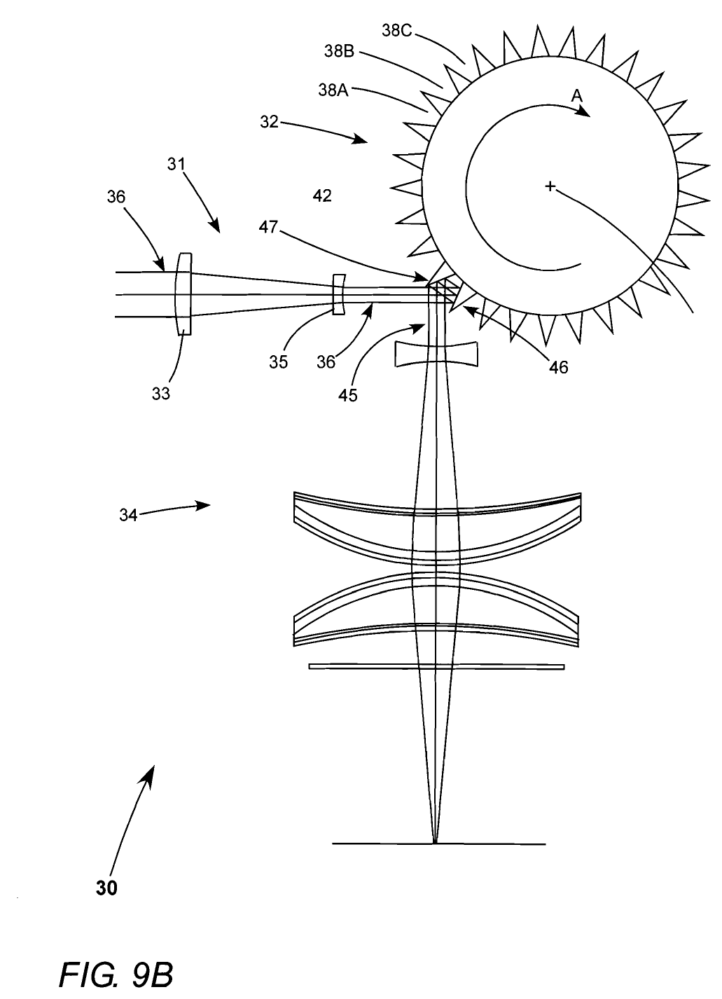

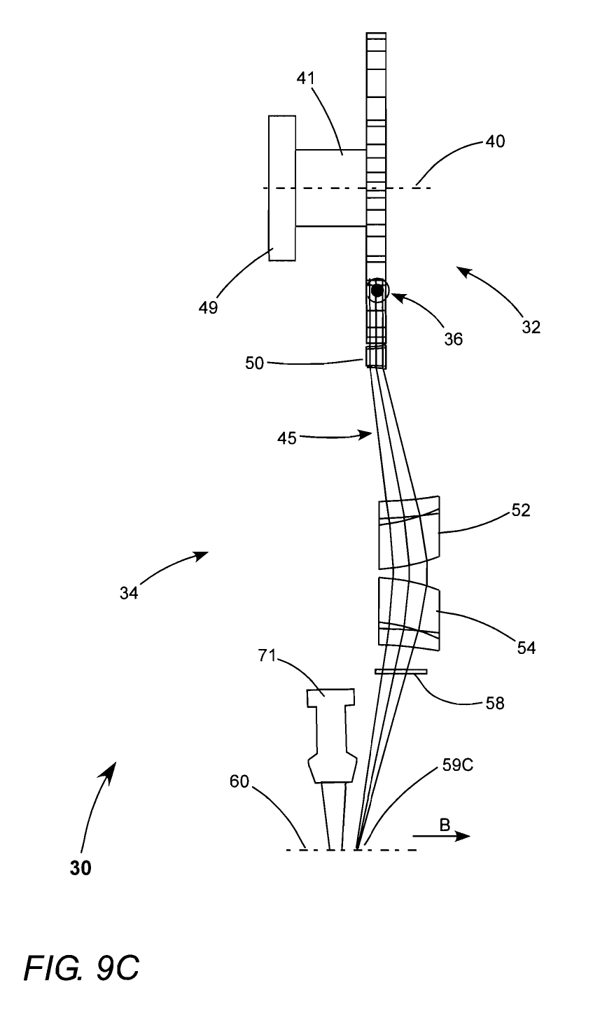

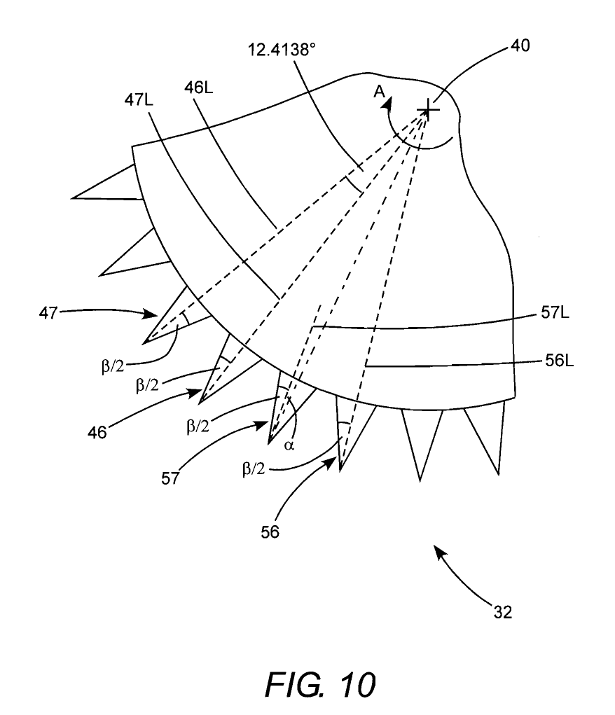

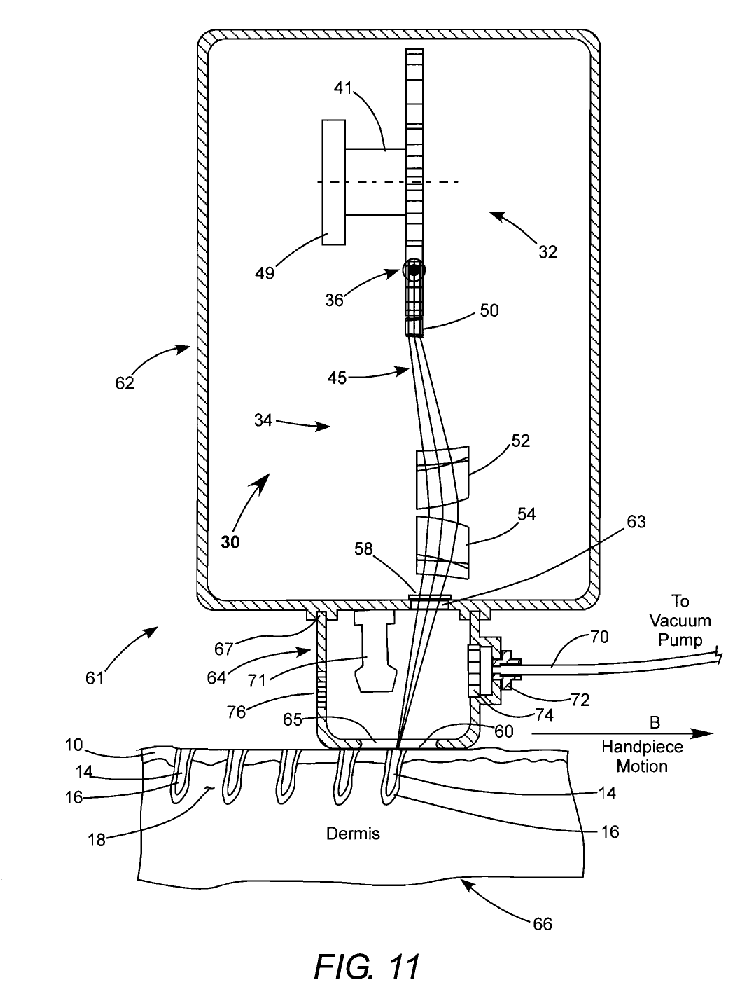

In any area being treated, ideally, all voids should be ablated simultaneously. However, apparatus capable of simultaneously ablating an effective number of voids with appropriate spacing over a useful area of skin may not be practical or cost effective. Practically, the voids can be ablated sequentially, but because of the rapid onset of the healing process, it is preferable that sequential ablation of tissue to create the voids in an area being treated is completed in a time period less than about 60 minutes (min). It is preferable to create voids at a rate between about 10 Hz and 5000 Hz and more preferably at a rate between about 100 Hz and 5000 Hz, because this rate reduces the physician time for treatment. Increasing the treatment rate above 5000 Hz causes the laser and scanning systems to be more expensive and therefore less commercially desirable, even though they are technologically feasible using the apparatus presented here. One preferred example of apparatus in accordance with the present invention for providing rapid sequential delivery of optical pulses and immediately thereafter introducing stem cells and differentiation factor into the voids is described below with reference to FIG. 9A, FIG. 9B, FIG. 9C, FIG. 10, and FIG. 11. FIGS. 9A-C and FIG. 10 depict apparatus for ablating the voids and FIG. 10 depicts an applicator including the void-ablating apparatus and means for introducing the stem cells and differentiation factor into the voids.

Beginning with a description of the laser apparatus, FIG. 9A is a front elevation view schematically illustrating an ablation apparatus 130 including a scanner wheel 132 and a wide field projection lens 134. The scanner wheel is driven by a motor 149 via a hub 141 (see FIG. 9C). Scanner wheel 132 is arranged to receive an incident laser beam 136 lying substantially in the plane of rotation of the scanner wheel. In FIG. 9A beam 36 is represented by only a single principle ray. FIG. 9B and FIG. 9C are respectively front and side elevation views of apparatus in which beam 36 is represented by a plurality of rays.

Before being incident on the scanning wheel, beam 136 is compressed (see FIG. 9B) by a telescope 131 comprising a positive lens 133 and a negative lens 135. In this example, the scanner wheel divided into twenty nine sectors 138A, 138B, 138C, etc., which are arranged in a circle centered on the rotation axis 140 of the scanner wheel. The wheel, here, is assumed to rotate in a clockwise direction as indicated by arrow A. The incident laser beam 136 propagates along a direction that lies in the plane of rotation. Each sector 138 of scanner wheel 132 includes a pair of reflective elements, for example, reflective surfaces 142 and 143 for the sector that is indicated as being active. The surface normals of the reflective surfaces have a substantial component in the plane of rotation of the scanner wheel. In this example, the scanner wheel includes prisms 146, 147, etc. that are arranged in a circle. The faces of the prisms are reflectively coated and the reflectively coated surfaces of adjacent prisms, for example, reflective surfaces 142 and 143 from prisms 146 and 147, form the opposing reflective surfaces for a sector. Alternatively, the reflective surfaces can be metal surfaces that are polished to be smooth enough to cause sufficient reflectivity.

Each sector 138 deflects the incoming optical beam 136 by some angular amount. The sectors 138 are designed so that the angular deflection is approximately constant as each sector rotates through the incident optical beam 136, but the angular deflection varies from sector to sector. In more detail, the incident optical beam 136 reflects from the first reflective surface 132 on prism 146, and subsequently reflects from reflective surface 143 on prism 147 before exiting as output optical beam 145.

The two reflective surfaces 142 and 143 form a Penta mirror geometry. An even number of reflective surfaces that rotate together in the plane of the folded optical path has the property that the angular deflection of output beam 145 from input beam 136 is invariant with the rotation angle of the reflective surfaces. In this case, there are two reflective surfaces 142 and 143 and rotation of the scanner wheel 132 causes the prisms 146 and 147 and reflective surfaces 142 and 143 thereof to rotate together in the plane of the folded optical path. As a result, the output beam direction does not change as the two reflective surfaces 142 and 143 rotate through the incident optical beam 136. The beam can be focused at the treatment surface such that the beam does not walk across the surface during the scanning or the beam can be used at another plane such that the beam walks across the surface during the scanning due to the translation of the beam in a conjugate plane that translates into an angular variation during the scanning due to the rotation of the scanning wheel. The reflective surfaces 142 and 143 are self-compensating with respect to rotation of scanner wheel 32. Furthermore, as the reflective surfaces 142 and 143 are planar, they will also be substantially spatially invariant with respect to wobble of the scanner wheel.

As the scanner wheel rotates clockwise to the next sector 138 and the next two reflective surfaces, the angular deflection can be changed by using a different included angle between the opposing reflective surfaces. For this configuration, the beam will be deflected by an angle that is twice that of the included angle. By way of example, if the included angle for sector 138A is 45 degrees, sector 138A will deflect the incident laser beam by 90 degrees. If the included angle for sector 138B is 44.5 degrees, then the incident laser beam will be deflected by 89 degrees, and so on. In this example, different included angles are used for each of the sectors so that each sector will produce an output optical beam that is deflected by a different amount. However, the deflection angle will be substantially invariant within each sector due to the even number of reflective surfaces rotating together through the incident beam. For this example, the angular deflections have a nominal magnitude of 90 degrees and a variance of −15 to +15 degrees from the nominal magnitude. Beam 145 in extreme left and right scanning positions is indicated by dashed lines 45L and 45R respectively. Here again, in FIG. 9A beam 145 is represented by only a single principle ray, while FIG. 9B and FIG. 9B represent beam 145 by a plurality of rays.

Referring in particular to FIG. 10, in this example of scanner wheel 132, the apex angle of each prism is 32.5862 degrees, calculated as follows. Each sector 138 subtends an equal angular amount. Since there are twenty nine sectors, each sector subtends 360/29=12.4138 degrees. The two prisms 146 and 147 have the same shape and, therefore, the same apex angle β. Scanner wheel 132 is designed so that when the included angle is 45 degrees, the prisms 146 and 147 are positioned so that lines 147L and 146L that bisect the apex angle of prisms 146 and 147 also passes through the rotation axis 140. Accordingly, the design must satisfy an equation β/2+12.4138+β/2=45. Solving this equation yields an apex angle of β=32.5862 degrees.

The next prism 157 moving counterclockwise on scanner wheel 132 from prism 146 is tilted slightly by an angle +α so its bisecting line 157L does not pass through the center of rotation 140 of the scanner wheel. As a result, the included angle for the sector formed by prisms 146 and 157 is (β/2+α)+12.4138+β/2=45+α. The next prism 156 is once again aligned with the rotation center 140 (as indicated by bisecting line 56L), so the included angle for the sector formed by prisms 56 and 57 is (β/2−α)+12.4138+β/2=45−α. The next prism is tilted by +2α, followed by an aligned prism, and then a prism tilted by +3α, followed by another aligned prism, etc. This geometry is maintained around the periphery of the scanner wheel. This specific arrangement produces twenty nine deflection angles that vary over the range of −15 degrees to +15 degrees relative to the nominal 90 degree magnitude. Note that this approach uses an odd number of sectors where every other (approximately) prism is aligned and the alternate prisms are tilted by angles α, 2α, 3α, etc. In an alternate embodiment, the surface on which beam 136 is incident has zero tilt and all tilt is taken up in the reflective surface on the second facet.

Wide field lens 134, here includes optical elements 150, 152, and 154, and an output window 158. In the lens depicted in FIGS. 9A-C the optical elements are assumed to made from zinc selenide which has excellent transparency for 10.6-micrometer radiation. Those skilled in the art will recognize that other IR transparent materials such as zinc sulfide (ZnS) or germanium (Ge) may be used for elements in such a lens with appropriate reconfiguration of the elements. Optical elements 152, 154, and 156 are tilted off axis spherical elements. Lens 134 focuses exit beam 145 from scanner wheel 132 in a plane 160 in which skin to be treated would be located. Lens 134 focuses exit beam 145 at each angular position that the beam leaves scanner wheel 132. This provides a line or row sequence of 29 focal spots (one for each scanning sector of the scanner wheel) in plane 160. In FIG. 9A three of those spots are designated including an extreme left spot 159L, a center spot 159C and an extreme right spot 159R. The remaining 26 spots (not shown) are approximately evenly distributed between spots 159L, 159C, and 159R. Another line of focal spots can be produced by moving apparatus 130 perpendicular to the original line as indicated in FIG. 9C by arrow B.

Referring in particular to FIG. 9C, the tilted off-axis spherical elements 150, 152 and 154 are arranged such that beam 145 is first directed, (by bi-concave negative) lens element 150, away from the plane of rotation of the scanner wheel. Elements 152 and 154 (positive meniscus elements) then direct the beam back towards the plane or rotation, while focusing the beam, such that the focused beam is incident non-normally (non-orthogonally) in plane 160, i.e., normal to skin being treated. One particular of this non-normal incidence of beam 145 on the skin is that window 158 and optical element 154 are laterally displaced from the focal point and are removed from the principal path of debris that may be ejected from a site being irradiated. Another advantage is that a motion sensor optics for controlling firing of the laser in accordance with distance traveled by the apparatus, for example, an optical mouse or the like, designated in FIG. 9C by the reference numeral 171, may be directed close to the point of irradiation. This is advantageous for control accuracy. As far as the actual treatment is concerned, it is not believed that there is any advantage of non-orthogonal compared with non-orthogonal (normal incidence) irradiation.

Those skilled in the art will recognize that it is not necessary that all sectors of the scanner wheel have a different deflection angle. Prisms of the scanning wheel can be configured such that groups of two or more sectors provide the same deflection angle with the deflection angle being varied from group to group. Such a configuration can be used to provide fewer voids in a row with increased spacing therebetween. It is also not necessary that deflection angle be increased or decreased progressively from sector to sector. It is preferred in that pulsed operation of the laser providing beam 136, that the PRF of the laser is synchronized with rotation of the scanner wheel such that sequential sectors of the wheel enter the path of beam 136 to intercept sequential pulses from the laser.

It should be noted here that apparatus 130 including scanner wheel 132 and focusing lens 134 is one of several combinations of scanning and focusing devices that could be used for carrying out the method of the present invention and the description of this particular apparatus should not be construed as limiting the invention. By way of example, different rotary scanning devices and focusing lenses are described in U.S. patent application Ser. No. 11/158,907, filed Jun. 20, 2005, the complete disclosure of which is hereby incorporated by reference. Galvanometer-based reflective scanning systems can also be used to practice this invention and have the advantage of being robust and well-proven technology for laser delivery. Scanning rates with a galvanometer-based reflective scanning systems, however, will be more limited than with the a scanner such as scanning wheel 132 described above, due to the inertia of the reflective component and the changes of direction required to form a scanning pattern over a substantial treatment area. Other scanner systems can be used and are well known in the art.

FIG. 11 schematically illustrates one embodiment of a handpiece 161 or applicator in accordance with the present invention including an example of above described apparatus 130. Handpiece 161 is depicted irradiating a fragment 166 of skin being treated. The handpiece is moved over the skin being treated, as indicated by arrow B, with tip 164 in contact with the skin. The irradiation provides parallel spaced-apart rows of above-described spaced-apart voids 14, only end ones of which are visible in FIG. 8. Spacing between the rows of spots may be narrower or broader than that depicted in FIG. 8, the spacing, here, being selected for convenience of illustration. Control of the row spacing can be affected by controlling delivery of the laser beam by optical motion sensor 171, or alternatively a mechanical motion sensor (mechanical mouse), as is known in the art. A description of such motion sensing and control is not necessary for understanding principles of the present invention and accordingly is not presented here. Descriptions of techniques for controlling delivery of a pattern of laser spots are provided in U.S. patent application Ser. No. 10/888,356 entitled “Method and Apparatus for fractional photo therapy of skin” and No. 11/020,648 entitled “Method and apparatus for monitoring and controlling laser-induced tissue treatment,” the complete disclosures of which are hereby incorporated herein by reference.

In a preferred method of operation, apparatus 130 is housed in handpiece or applicator 161 including a housing 162 to which is attached an open-topped, removable tip 164, which is attached to the housing via slots 167. Pins and/or screws can also be used for this purpose. When tip 164 is attached to housing the tip is divided into two chambers 182 and 184 having no gas-passage therebetween. An aperture 163 in housing 162 is covered by window 158 such that optical access to chamber 182 is provided while preventing gas passage between the housing and chamber 182. In use, the base of the tip makes a reasonable gas-tight seal with the skin.

Laser beam 136 is directed into housing 162 via an articulated arm (not shown). Articulated arms for delivery infra red laser radiation are well known in the art. One preferred articulated arm is described in U.S. Patent Application No. 60/752,850 filed Dec. 21, 2005 entitled “Articulated arm for delivering a laser beam,” the complete disclosure of which is hereby incorporated herein by reference. The focused beam 145 from lens 134 exits housing 162 via exit window 158, (here attached to the housing) and via aperture 163 in the housing, then passes through chamber 182 of tip 164 exiting via aperture 165 therein. A vacuum pump (not shown) is connected to removable tip 164 via a hose or tube 170. Tube 170 is connected to tip 164 via a removable and replaceable adaptor 172. Operating the vacuum pump with tip 164 in contact with the skin creates negative pressure (partial vacuum) inside the tip. This withdraws smoke resulting from the laser ablation from the path of the laser beam, and draws debris products of the ablation away from window 158 in the housing. A filter element 174 in a wall of tip 164 prevents debris from being drawn into vacuum hose 170 and eventually into the pump.

The arrangement of the tip provides that, when the vacuum pump is operated, there is also negative pressure created in any void that is under the aperture. The seal of the base of the tip to the skin retains the negative pressure in voids over which the tip has passed. Chamber 184 of tip 164 serves as a reservoir for a mixture of stem cells and differentiating medium 188. A channel though the tip, from chamber 184 through the base of the tip, allows a flow of the stem-cell mixture into the voids. An aperture 190 through the tip allows gas to enter chamber 184 to assist in the free flow of the mixture through channel 192. It is also possible to supply positive pressure through such an aperture to further encourage flow of the mixture, where pressure is measured relative to the ambient pressure outside of the apparatus. Alternatively, the stem cells can be applied topically following laser irradiation without the assistance of vacuum or positive pressure.

### EXAMPLES

Having now generally described the invention, the same may be more readily understood through the following reference to the following examples. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

### Micropore Channel Creation

Freshly excised human skin samples was irradiated with a 30 W, 10.6 μm CO₂ laser at varying pulse energies. The laser beams carried a near diffraction limited 1/e² Gaussian spot size of approximately 120 μm, with pulse energies ranging from 8 to 20 mJ that are delivered through an apparatus capable of a repetition rate up to 1500 spots/second.

The skin was heated on a digital hot plate (Cole-Parmer Instrument Co., Vernon Hills, Ill.), and the skin surface temperature was measured with a Mintemp MT4 infrared probe (Raytek Corporation, Santa Cruz, Calif.). The laser treatment was initiated when the skin surface reached a temperature of 98±3° F. The laser handpiece was translated at a specific velocity by using a precision linear stage driven by an ESP 300 motion controller (Newport Co., Irvine, Calif.). The firing rate of the laser was automatically adjusted by the laser handpiece to produce a specific density of lesions. A single pass was made at a constant velocity of 1.0 cm/s and spot density of 400 microscopic ablative treatment zones per cm² creating an interlesional distance of approximately 500 μm. The voids thus created were about 200 μm to 4 mm in depth.

### Example 2

### Application of a Vitamin C and E Formulation to Skin During and Following Laser Skin Treatment

A topical vitamin C and E solution containing L-ascorbic acid 15% (VWR International, West Chester, Pa.), ferulic acid 0.5%, and vitamin E 1% buffered to a pH of 3.2±0.2 with triethanolamine is prepared as described by Lin et al, J Invest Dermatol. 2005 October; 125(4):826-32. The formulation is applied to the face of a subject immediately prior to fractional laser resurfacing treatment of the face. Within 1 minute of completion of the laser treatment, the formulation is reapplied. The patient is instructed to continue applying the formulation to the treated region of the skin two times daily for the next 5 days.

### Example 3

### Application of a Vitamin C Composition to Skin Following Laser Treatment

A cosmetically elegant multiple phase oil/water/oil emulsion of vitamin C is prepared as described by Farahmand et al, Pharm Dev Technol. 2006; 11(2):255-61. A vascular lesion in the patient's skin is treated using a fractional ablative laser technique. Following the laser treatment, the vitamin C composition is applied to the treated region of skin. The patient is instructed to continue applying the formulation to the treated region of skin three times daily for the next week.

### Example 4

### Application of a Vitamin C, Green Tea Extract, Bioflavinoid Complex and Aloe Vera Mask to Skin Following Laser Treatment

A cosmetically elegant hydrogel mask is prepared and infused with ascorbic acid, green tea extract, a bioflavinoid complex, and aloe vera extract. The patient's facial skin is treated using fractional ablative laser radiation to treat wrinkles and photoaging of the skin. Immediately following the laser treatment, the infused hydrogel mask is activated and placed on the patient's skin for 30 minutes to 2 hours. The patient is instructed to continue using the masks at home one to two times daily for the next two weeks.

Those skilled in the art may devise other active substances, compositions and methods of applying them without departing from the spirit and scope of the present invention.

### Example 5

### Application of a Vasoconstrictor Composition to Skin Prior to and Following Laser Treatment

A liquid formulation suitable for spraying on the skin containing 0.25% phenylephrine is prepared as described in Kratz and Danon, Injury. 2004 November; 35(11):11096-101. Thirty minutes prior to treating the patient's skin with an ablative fractional laser treatment to rejuvenate the skin, the phenylephrine spray is applied to the skin. The skin is then treated using fractional ablative laser radiation. Following the laser treatment, the phenylephrine formulation is again applied to the treated region of skin. The formulation reduces the amount of exudate secreted by the skin during treatment, and reduces skin inflammation following the treatment.

### Example 6

### Application of an Antihistamine Composition to Skin Prior to and Following Laser Treatment

A gel formulation suitable for applying to the skin containing 1% centirizine dinitrate is prepared. Fifteen minutes prior to treating the patient's skin with an ablative fractional laser treatment to rejuvenate the skin, the centirizine gel is applied to the skin. The skin is then treated using fractional ablative laser radiation. Following the laser treatment, the centirizine gel is again applied to the treated region of skin. The gel reduces the amount of exudate secreted by the skin during treatment, and reduces skin inflammation following the treatment.

### Example 7

### Application of an Antioxidant Hydrogel Mask to Skin Following Laser Skin Resurfacing

A cosmetically elegant hydrogel mask is prepared and infused with antioxidants commonly used in topical cosmetic compositions. The patient's facial skin is resurfaced using ablative CO₂ laser radiation delivered in a fractional manner (i.e., delivered in a manner so as to produce a plurality of micropore channels in the treated region of skin) to treat wrinkles and photoaging of the skin. Immediately following the laser treatment, the infused hydrogel mask is activated and placed on the treated region of the patient's skin for 30 minutes to 2 hours. The patient is instructed to continue using the masks at home one to two times daily for the next two weeks to one month. Use of the masks following the treatment significantly reduces the incidence of side effects such as, for example, edema and erythema, and increase the rate of healing of the treated region of skin.

All printed patents and publications referred to in this application are hereby incorporated herein in their entirety by this reference.

What is claimed is:

1. A method of delivering vitamin C into the skin for local treatment of the skin of a subject in need thereof, the method comprising:
irradiating skin with laser irradiation to form a plurality of micropore channels wherein the micropore channels extend into a dermal layer of the skin; and
applying vitamin C into the micropore channel.

2. The method of claim 1, wherein the vitamin C is applied 1 minute after the formation of the plurality of micropore channels.

3. The method of claim 1, wherein the vitamin C is applied 1 hour after the formation of the plurality of micropore channels.

4. The method of claim 1, wherein the vitamin C is applied 1 day after the formation of the plurality of micropore channels.

5. The method of claim 1, wherein the plurality of micropore channels are elongated.

6. The method of claim 1, wherein viable tissue separates the plurality of elongated micropore channels.

7. The method of claim 1, wherein the vitamin C comprises a cosmetically effective amount of a form of vitamin C in a cosmetically acceptable carrier.

8. An apparatus for treating skin, the apparatus comprising:
a handpiece movable over skin wherein the handpiece is arranged to receive an optical beam and focus the optical beam at a plurality of spaced-apart locations on the skin thereby creating a plurality of voids in the skin for the deposition of a composition.

9. The apparatus of claim 8, further comprising an applicator arranged to deposit a composition in the voids following the formation of the voids.

10. The apparatus of claim 9, wherein the applicator further comprises a removable tip that attaches to the handpiece.

11. The apparatus of claim 8, wherein the composition is a cosmetically effective amount of vitamin C in a cosmetically acceptable carrier.

12. The apparatus of claim 8, wherein the composition is a cosmetically effective amount of an antioxidant.

13. The apparatus of claim 8, wherein viable tissue separates the plurality of voids.

14. The apparatus of claim 13, wherein the viable tissue separating any two voids is between 50 and 500 μm at its narrowest point.

15. The apparatus of claim 8, wherein the voids are created with a density of 200-4000 voids per cm² in a single pass.

16. The apparatus of claim 8, wherein the voids are created at a rate of 10 to 5000 per second.

17. The apparatus of claim 8, wherein the voids are created at a rate of 100 to 5000 per second.

18. The apparatus of claim 8, wherein the pulse energy is 5 to 40 mJ per void.

19. The apparatus of claim 8, further comprising a scanner.

20. The apparatus of claim 19, wherein the scanner comprises a reflective rotating scanner.

21. The apparatus of claim 19, wherein the scanner comprises one or more galvanometer scanners.

22. The apparatus of claim 8, wherein the optical beam is emitted by a laser.

23. The apparatus of claim 22, wherein the laser is a CO₂ laser with a wavelength of about 10.6 μm.

24. The apparatus of claim 8, wherein the optical beam has an absorption coefficient in water of about 100 to 12,300 cm⁻¹.

25. The apparatus of claim 8, wherein the optical beam has an absorption coefficient in water of about 500 to 1000 cm⁻¹.

26. The apparatus of claim 8, wherein the voids are about 200 μm to 4 mm in depth.

27. The apparatus of claim 8, further comprising a vacuum that removes debris that is removed from the skin during creation of the voids.

28. The apparatus of claim 8, further comprising a system that creates a positive pressure in a chamber containing the composition.

29. The apparatus of claim 8, wherein the voids are elongated.

30. A kit for use with a laser delivery system comprising:
a handpiece movable over skin wherein the handpiece is arranged to receive a laser beam and focus the laser beam at a plurality of spaced-apart locations on the skin thereby creating a plurality of voids in the skin for the deposition of a composition, and
an applicator arranged to deposit a composition in the voids following the formation of the voids.

31. A method of delivering an active substance into the skin for local treatment of resurfaced skin, the method comprising:
selecting a region of skin in need of resurfacing treatment and treatment with an active substance;
irradiating skin with laser irradiation to ablate tissue to form a plurality of micropore channels wherein the micropore channels extend into a dermal layer of the skin, thereby resurfacing the region of skin and increasing permeability of the region of skin to an active substance; and
applying the active substance to the region of skin.

32. The method of claim 31, wherein the laser irradiation is provided by a CO₂ laser system which delivers the irradiation in a fractional manner.

33. The method of claim 31, wherein the active substance is an antioxidant composition.

34. The method of claim 31, wherein the active substance is applied in the form of a hydrogel mask.

35. The method of claim 31, wherein the active substance is applied immediately following the laser irradiation.

36. The method of claim 31, wherein the active substance is applied repeatedly up to one month following the laser irradiation.
